(19) United States
(12) Patent Application Publication
(10) Pub. No.: US 2018/0280385 A1
(43) Pub. Date: Oct. 4, 2018

(54) NINTEDANIB FOR USE IN METHODS FOR THE TREATMENT OF MUSCULAR DYSTROPHY

(71) Applicant: Boehringer Ingelheim International GmbH, Ingelheim am Rhein (DE)

(72) Inventors: Stefan Ludwig Michael WOLLIN, Bad Waldsee (DE); Jorge Alberto DIAZ MANERA, Barcelona (ES)

(21) Appl. No.: 15/936,481

(22) Filed: Mar. 27, 2018

(30) Foreign Application Priority Data
Mar. 28, 2017 (EP) 17 163 355.5

(51) Int. Cl. A61K 31/496; A61K 9/20; A61K 9/48; A61P 21/00

(52) U.S. Cl. CPC A61K 31/496; A61P 21/00; A61K 9/48; A61K 9/20

(57) ABSTRACT

The invention relates to the use of tyrosine kinase inhibitors, selected from nintedanib and pharmaceutically acceptable salts thereof, for the treatment of muscular dystrophy.

### FIELD OF THE INVENTION

This invention relates to tyrosine kinase inhibitors, selected from nintedanib and pharmaceutically acceptable salts thereof, for use in methods for the treatment of muscular dystrophy. In addition, the invention relates to pharmaceutical compositions comprising said inhibitors and to methods for the treatment of muscular dystrophy with said inhibitors or compositions.

### BACKGROUND OF THE INVENTION

Muscular dystrophy (MD) is a group of muscular diseases that lead to weakening and progressive degeneration of muscles. The most common form of MD is Duchenne muscular dystrophy (DMD), an X-linked recessive neuromuscular disorder, resulting eventually in premature death (Mah, Neuropsychiatr Dis Treat 2016, 12, 1795-1807). The average life expectancy for individuals afflicted with DMD is around 25 years.

DMD is caused by a mutation in the gene coding for the protein dystrophin which is an important component within muscle tissue that provides structural stability.

Early symptoms of DMD include muscle weakness associated with loss of muscle mass (wasting), pseudohypertrophy and low endurance. As the condition progresses, muscle tissue experiences wasting and is eventually replaced by fat and fibrotic tissue (fibrosis). Later symptoms may include abnormal bone development leading to skeletal deformities, including curvature of the spine (neuromuscular scoliosis), and loss of movement, eventually leading to paralysis. Intellectual impairment may or may not be present.

One of the significant factors causing morbidity and mortality in patients with DMD and neuromuscular scoliosis is the progressive loss in lung function (Kennedy et al., Thorax 1995, 50, 1173-1178). DMD affects the pulmonary function because of respiratory muscle weakness, impaired mucociliary clearance and impaired coughing, a reduction in the compliance of the lung and the chest wall and a restriction by the scoliosis (Gozal, Pediatr Pulmonol 2000, 29, 141-150).

The mechanisms that lead to the degeneration of skeletal muscle in patients with muscle dystrophies have been extensively studied (Kharraz et al., Biomed Res Int 2014, 2014, 965631). One of the processes that run in parallel during muscle degeneration is the proliferation of fibro-adipose progenitor cells and their secretion of extracellular matrix components, which leads to an expansion of fibrous tissue.

Platelet-derived growth factors (PDGFs) constitute a family of growth factors associated with multiple cellular processes such as proliferation, chemotaxis and cell differentiation. These molecules have been related with fibrosis of several tissues in humans. A role of the PDGF signalling cascade in muscle fibrosis is suggested (Bonner, Growth Factor Rev 2004, 15, 255-273) by the increased PDGF-AA and -BB expression in damaged muscle fibers in vivo and in affected muscles of DMD patients. Also, PDGF-AA activates fibroblast proliferation and the expression of the different components of the extracellular matrix in vitro (Bonner, Growth Factor Rev 2004, 15, 255-273). Increased PDGF receptor-a activation disrupts connective tissue development and drives systemic fibrosis (Olson et al., Dev Cell 2009, 16, 303-313).

Available drug treatments of DMD are based on exon skipping (eteplirsen) or bypassing nonsense mutations in the dystrophin coding gene (ataluren).

The tyrosine kinase inhibitor imatinib had shown efficacy in an animal model of DMD (Bizario et al., J Neuroimmunol 2009, 212, 93-101; Huang et al., FASEB J 2009, 23, 2539-2548; Ito et al., Neuromuscular Disorders 2013, 23, 349-56), but the results of clinical trials of imatinib in patients with fibrotic disorders (Daniels et al., Am J Respir Crit Care Med 2010, 181, 604-610) did not warrant the continuation of the clinical program beyond phase II.

Nintedanib, the compound of formula A,

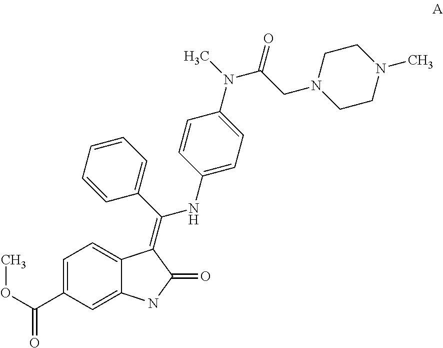

is an innovative compound having valuable pharmacological properties, e.g. for the treatment of oncological diseases, immunologic diseases or pathological conditions involving an immunologic component, or fibrotic diseases.

Nintedanib is described in WO 01/27081. WO 2004/013099 discloses its monoethanesulphonate salt; further salt forms are presented in WO 2007/141283.

Pharmaceutical dosage forms comprising nintedanib are disclosed in WO 2009/147212 and in WO 2009/147220.

The use of nintedanib for the treatment of immunologic diseases or pathological conditions involving an immunologic component is described in WO 2004/017948, the use for the treatment of oncological diseases is described in WO 2004/096224 and the use for the treatment of fibrotic diseases is described in WO 2006/067165.

Nintedanib is a highly potent, orally bioavailable inhibitor of vascular endothelial growth factor receptors (VEGFRs), platelet-derived growth factor receptors (PDGFRs) and fibroblast growth factor receptors (FGFRs). It binds competitively to the adenosine triphosphate (ATP) binding pocket of these receptors and blocks intracellular signalling. In addition, nintedanib inhibits Fms-like tyrosine-protein kinase 3 (Flt 3), lymphocyte-specific tyrosine-protein kinase (Lck), tyrosine-protein kinase lyn (Lyn) and proto-oncogene tyrosine-protein kinase src (Src) (Hilberg et al., Cancer Res. 2008, 68, 4774-4782).

### SUMMARY OF THE INVENTION

In a first aspect, the present invention relates to a tyrosine kinase inhibitor, selected from nintedanib and pharmaceutically acceptable salts thereof, for use in a method for the treatment of muscular dystrophy.

In a second aspect, the present invention relates to a pharmaceutical composition comprising one or more of said inhibitors and one or more pharmaceutically acceptable excipients for use in a method for the treatment of muscular dystrophy.

In a third aspect, the present invention relates to a method for the treatment of muscular dystrophy in a patient in need thereof, the method being characterized in that one or more of said inhibitors is administered to the patient.

In a forth aspect, the present invention relates to the use of one or more of said inhibitors in the manufacture of a medicament for the treatment of muscular dystrophy in a patient in need thereof.

In another aspect, the present invention relates to a method for treating muscular dystrophy comprising administering a pharmaceutically effective amount of nintedanib, or a pharmaceutically acceptable salt thereof, to a patient in need thereof.

In another aspect, the present invention relates to a method for treating muscular dystrophy consisting essentially of administering a pharmaceutically effective amount of nintedanib, or a pharmaceutically acceptable salt thereof, to a patient in need thereof.

In another aspect, the present invention relates to a method for treating muscular dystrophy consisting of administering a pharmaceutically effective amount of nintedanib, or a pharmaceutically acceptable salt thereof, to a patient in need thereof.

Other aspects of the present invention will become apparent to the person skilled in the art directly from the foregoing and following description.

### BRIEF DESCRIPTION OF THE DRAWINGS

FIG. 1:

Amplitude of the compound muscle action potential (CMAP) of control wild type mice (WT), non-treated mdx mice (Mdx) and nintedanib-treated mdx mice (Mdx+Nintedanib), determined in the tibialis anterior muscle (A), Gastrocnemius (B) and Plantar muscle (C). ** p<0.01, **** p<0.0001 versus WT, # p<0.05 versus Mdx.

FIG. 2:

Percentage of small (A), medium (B) and large (C) motor unit action potentials (MUAPs) of control wild type mice (WT), non-treated mdx mice (Mdx) and nintedanib-treated mdx mice (Mdx+Nintedanib). * p<0.05 versus WT.

FIG. 3:

Quantification of collagen VI-stained tissue sections of diaphragms (A), quadriceps (B) and tibialis anterior (C) from control wild type mice (WT), non-treated mdx mice (Mdx) and nintedanib-treated mdx mice (Mdx+Nintedanib). ROI=region of interest, * p<0.05 versus Mdx.

FIG. 4:

Quantification of collagen I, normalized to alpha-tubulin levels, in tissue of quadriceps (A), diaphragm (B) and tibialis anterior (C) from control wild type mice (WT), non-treated mdx mice (Mdx) and nintedanib-treated mdx mice (Mdx+Nintedanib) by western blotting analysis. * p≤0.05, ** p≤0.01 versus Mdx.

FIG. 5:

Longest suspension time in a so called “hanging wire” test, recorded in mdx mice before the start of the treatment period and after 7 weeks of treatment with nintedanib compared to untreated mdx mice.

FIG. 6:

“Fall and reaches” score in a so called “hanging wire” test, recorded in mdx mice before the start of the treatment period and after 7 weeks of treatment with nintedanib compared to untreated mdx mice.

### GENERAL TERMS AND DEFINITIONS

Terms not specifically defined herein should be given the meanings that would be given to them by one of skill in the art in light of the disclosure and the context. As used in the specification, however, unless specified to the contrary, the following terms have the meaning indicated and the following conventions are adhered to.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, and commensurate with a reasonable benefit/risk ratio.

As used herein, “pharmaceutically acceptable salt” refers to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like.

The terms “treatment” and “treating” as used herein embrace both therapeutic, i.e. curative and/or palliative, and preventive, i.e. prophylactic, treatment.

Therapeutic treatment refers to the treatment of patients having already developed one or more of said conditions in manifest, acute or chronic form. Therapeutic treatment may be symptomatic treatment in order to relieve the symptoms of the specific indication or causal treatment in order to reverse or partially reverse the conditions of the indication or to stop or slow down progression of the disease.

Preventive treatment (“prevention”) refers to the treatment of patients at risk of developing one or more of said conditions, prior to the clinical onset of the disease in order to reduce said risk.

The terms “treatment” and “treating” include the administration of one or more active compounds in order to prevent or delay the onset of the symptoms or complications and to prevent or delay the development of the disease, condition or disorder and/or in order to eliminate or control the disease, condition or disorder as well as to alleviate the symptoms or complications associated with the disease, condition or disorder.

The term “therapeutically effective amount” means an amount of a compound of the present invention that (i) treats or prevents the particular disease or condition, (ii) attenuates, ameliorates, or eliminates one or more symptoms of the particular disease or condition, or (iii) prevents or delays the onset of one or more symptoms of the particular disease or condition described herein.

### DETAILED DESCRIPTION OF THE INVENTION

The present invention allows for an efficient treatment of patients with muscular dystrophy (MD) with manageable systemic side effects by administration of tyrosine kinase inhibitors, selected from nintedanib and pharmaceutically acceptable salts thereof.

In a first aspect of the present invention, it is found that nintedanib is efficacious in an animal model of Duchenne muscular dystrophy (DMD).

The mdx mouse is a well characterized and widely used animal model for drug tests for DMD (Bulfield et al., Proc Natl Acad Sci USA 1984, 81, 1189-92). Mdx mice spontaneously develop a pathology resembling aspects of the human disease. They present cycles of degeneration and regeneration in the limb muscles and a progressive degeneration and fibrosis in the diaphragm (Coirault et al., J Appl Physiol 2003, 94, 1744-50). The first wave of degeneration peaks in the fourth week of life. At day 21, muscular lesions can be observed, including myofiber necrosis, leaking myofibers as a consequence of injuries to the sarcolemma, high levels of serum creatine kinase, and the presence of large areas of inflammation, connective tissue, and fat (Collins and Morgan, Int J Exp Pathol 2003, 84, 165-72; Grounds et al., Neurobiol Dis 2008, 31, 1-19). Compulsory physical activity can be applied to exacerbate the muscular degeneration during this phase (De Luca et al., Am J Pathol 2005, 166, 477-89; Grounds et al., Neurobiol Dis 2008, 31, 1-19).

The treatment of the mdx mice with nintedanib started at an age of 10 months in the 1^{st} study and at 6 weeks in a 2^{nd} study. Treatment duration was 4 weeks in the 1^{st} study and 7 weeks in the 2^{nd} study. Nintedanib was used at a dose of 60 mg/kg once daily administered intragastrically via gavage in both studies.

In the 1^{st} study, 7 mdx mice were treated with nintedanib, 5 mdx mice with the vehicle as placebo and 5 C57Bl6/mice (=wild type=WT) were used as unaffected, untreated control animals. At the end of the 1^{st} study, electrophysiological examinations using electroneurography and electromyography and a histological analysis were performed. In addition, the level of collagen I expression was investigated using western blot in 4 mice of each test group.

In the 2^{nd} study, 7 mdx mice were treated with nintedanib, 5 mdx mice with the vehicle as placebo and no WT control was included as unaffected, untreated control animals. At the end of the 2^{nd} study, a hanging wire test was performed.

Treatment with nintedanib resulted in improved electroneurography and electromyography as compared to untreated mdx mice:

In mdx mice, the electroneurography studies showed that the amplitude of the motor evoked potentials in the tibialis anterior muscle (FIG. 1A), the Gastrocnemius (FIG. 1B) and the Plantar muscle (FIG. 10) was reduced at the end of the study compared to WT animals. In the group of the nintedanib-treated mdx mice, an increase in the amplitude of the motor evoked potentials in all nerves studied was detected in comparison to non-treated mdx mice (FIG. 1A-C). The differences reached statistical significance in the tibialis anterior (FIG. 1A).

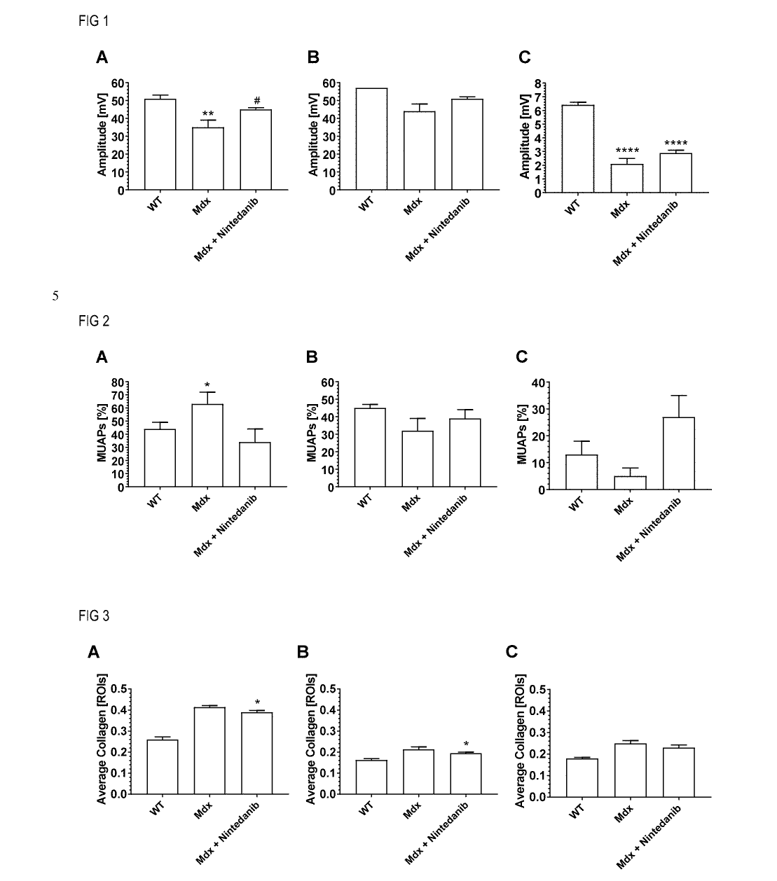

In the electromyography the percentage of small, medium and large motor unit action potentials (MUAPs) were recorded (Gordon et al., Can J Physiol Pharmacol 2004, 82, 645-661). The percentage of small MUAPs significantly increased in mdx mice (FIG. 2A) and the percentage of large MUAPs showed a trend towards a reduction (FIG. 2C) compared to WT animals. The nintedanib treated animals showed a normalized percentage of small MUAPs and an increase in large MUAPs indicating a structural improvement of the muscle fibers (FIG. 2A-C).

The histological analysis revealed that treatment with nintedanib resulted in reduced muscular fibrosis as compared to untreated mdx mice:

At the end of the 1^{st} study, the diaphragm from non-treated mdx mice had increased fibrotic tissue areas, compared to WT mice, and inflammatory infiltrates. The diaphragm from nintedanib-treated mdx mice had also dystrophic features, but the fibrotic area was clearly reduced and inflammatory infiltrates were not observed.

The fibrotic area of the muscles was assessed using immunofluorescent staining for collagen VI (Col VI). 8 photographs from different muscle sections of the quadriceps, tibialis anterior and diaphragm were randomly taken. The fluorescent area was analyzed using the software package Image J. The mean fibrotic area stained for Col VI on the diaphragma in non-treated 10 months old mdx mice was of 57.8% while it was reduced to 50.8% in nintedanib-treated animals. The reduction of 7% by nintedanib treatment was statistically significant (p<0.001) (FIG. 3A).

Similar results were also observed in Col VI-stained tissue sections of the quadriceps (FIG. 3B) and the tibialis anterior (FIG. 3C): In the quadriceps sections the Col VI staining was reduced from 25.4% in non-treated mdx mice to 23.1% in nintedanib-treated mdx mice (p=0.03). Although there was a tendency, the difference observed in Col VI-stained tibialis anterior sections did not reach statistical significance (21.4% in non-treated mdx mice versus 18.9% in nintedanib-treated mdx mice, p=0.06).

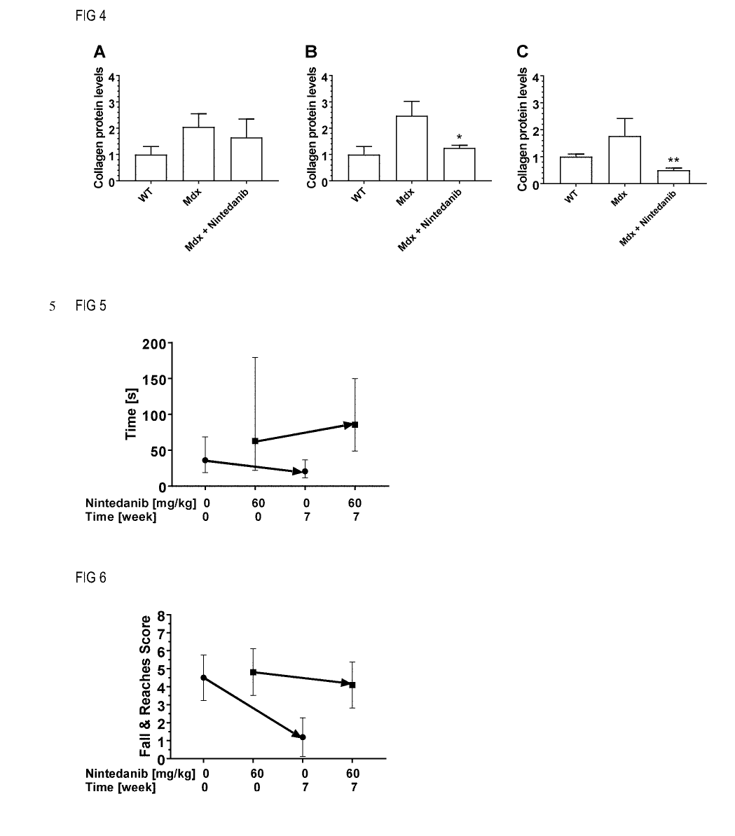

Also, a statistically significant decrease in collagen I (Col I) expression in the diaphragm and tibialis anterior of nintedanib treated mdx mice compared with mdx mice was observed by western blotting analysis (FIG. 4).

A reduction in muscle fibrosis as demonstrated by the reduced extracellular matrix production (Col VI staining and Col I quantification) is indicative for attenuation of further deterioration of the muscle function.

In the 2^{nd} study a so called “hanging wire” test was performed (see section Examples and Experimental Data; Rabl et al., BMC Neurosci 2017, 18, 22). This test can be used to assess global “subacute” muscle function and coordination over time in young and old mdx mice. The longest suspension time (LST) and the “fall and reaches” score (FRS) were recorded before the start of the treatment period and after 7 weeks of treatment with nintedanib.

In vehicle-treated mdx mice the LST showed a trend towards shorter times between 1^{st} assessment and 2^{nd} assessment 7 weeks later. In contrast, LST increased in nintedanib-treated mdx mice (FIG. 5).

Similarly, the FRS decreased during the 7 week observation period in the vehicle-treated mdx mice, but remained nearly constant in the nintedanib-treated animals (FIG. 6).

It can be assumed that the demonstrated pre-clinical effects will translate into a clinical improvement of the skeletal muscle strength and lung function in patients with DMD or similar muscular dystrophies if treated with nintedanib, in particular chronically.

According to one embodiment of the first aspect of the present invention, a tyrosine kinase inhibitor, selected from nintedanib and pharmaceutically acceptable salts thereof, preferably nintedanib in the form of its monoethanesulfonate salt, is provided for use in a method for the treatment of MD.

According to another embodiment, said inhibitor is preferably provided for use in a method for the treatment of MD, selected from Duchenne MD, Becker MD, myotonic dystrophy, facioscapulohumeral MD, limb-girdle MD, Emery-Dreifuss MD, congenital MD, oculopharyngeal MD, sarcoglycanopathies and dysferlinopathies like Miyoshi myopathy, most preferably for the treatment of Duchenne MD.

In a second aspect of the present invention, it is found that pharmaceutical compositions of the above-mentioned inhibitors may be formulated that are suitable for the administration of therapeutically effective amounts of said inhibitors for the treatment of muscular dystrophies.

Suitable preparations for administering the active pharmaceutical ingredients of the present invention will be apparent to those with ordinary skill in the art and include for example tablets, pills, capsules, suppositories, lozenges, troches, solutions, syrups, elixirs, sachets, injectables, inhalatives and powders etc. Suitable tablets may be obtained, for example, by mixing one or more of the above-mentioned active pharmaceutical ingredients with known excipients, for example inert diluents, carriers, disintegrants, adjuvants, surfactants, binders and/or lubricants.

For instance, therapeutically effective doses of nintedanib, when applied orally to a patient in need thereof, may be in the range from 50 mg to 300 mg per day, preferably twice daily application of 25 mg, 50 mg, 100 mg or 150 mg, approximately 12 hours apart.

The actual therapeutically effective amount or therapeutic dosage will of course depend on factors known by those skilled in the art such as age and weight of the patient, route of administration and severity of disease. In any case, the active compound will be administered at dosages and in a manner which allows a therapeutically effective amount to be delivered based upon a patient's unique condition. Likewise, the determination of the necessity of dose adjustments, e.g. due to adverse reactions to the active pharmaceutical ingredient, and their putting into practice will be known to the one skilled in the art.

According to one embodiment of the second aspect of the present invention, a pharmaceutical composition is provided that comprises one or more tyrosine kinase inhibitors, selected from nintedanib and pharmaceutically acceptable salts thereof, preferably nintedanib in the form of its monoethanesulfonate salt, and one or more pharmaceutically acceptable excipients for use in a method for the treatment of MD.

According to another embodiment, the pharmaceutical composition is selected from compositions for oral administration, preferably from capsules and tablets, most preferably from capsules.

According to another embodiment, the pharmaceutical composition is preferably provided for use in a method for the treatment of MD, selected from Duchenne MD, Becker MD, myotonic dystrophy, facioscapulohumeral MD, limb-girdle MD, Emery-Dreifuss MD, congenital MD, oculopharyngeal MD, sarcoglycanopathies and dysferlinopathies like Miyoshi myopathy, most preferably for the treatment of Duchenne MD.

In a third aspect, the present invention relates to a method for the treatment of muscular dystrophy in a patient in need thereof with one or more of the above-mentioned inhibitors. Furthermore, the present invention relates to a method for the treatment of muscular dystrophy with one or more of the above-mentioned pharmaceutical compositions.

In a forth aspect, the present invention relates to the use of the above-mentioned inhibitor in the manufacture of a medicament for the above-mentioned method for the treatment of muscular dystrophy.

### Examples and Experimental Data

The following examples are for the purpose of illustration of the invention only and are not intended in any way to limit the scope of the present invention.

### A) The Use of Hanging Wire Tests to Monitor Muscle Strength and Condition Over Time

The hanging wire test is performed following the standard operating procedure described in “The use of hanging wire tests to monitor muscle strength and condition over time,” Maaike van Putten (author), DMD_M.2.1.004 (revised version of 29 Jun. 2016) issued by the TREAT-NMD network and available at www.treat-nmd.eu/downloads/file/sops/dmd/MDX/DMD_M.2.1.004.pdf (last visited on Mar. 16, 2018) Essentials of this procedure are reproduced in the following:

“1 Objective

The hanging wire tests [ . . . ] can be used to assess global “subacute” muscle function and coordination over time in young and old mdx mice. [ . . . ] The test is based on the latency of a mouse to fall off a metal wire upon exhaustion. With the use of several described experimental designs, the natural course of the disease or the efficacy of genetic or pharmacologic treatment strategies can be assessed. Despite mdx mice having a less severe phenotype than DMD patients, differences in hanging performance between wild type (C57BL/10ScSnJ) and dystrophic (mdx or mdx5Cv) mice can be seen and experimental interventions can improve hanging performances. [ . . . ]

4 Material

A 55 cm wide 2-mm thick metallic wire (can be either a plain wire or a multistranded twisted wire, possibly plastic-coated) is secured to two vertical stands. The wire must be tightly attached to the frame to avoid vibration or unwanted displacement of the wire while the investigator is handling the animals or during the measurements, since these unwanted effects would interfere with the animal's performance. The wire is maintained 35 cm above a layer of bedding material to prevent injury to the animal when it falls down. Animals: Mice as young as four weeks, and as old as approximately 19 months of age have been reliably evaluated with this test, but the test might also be applicable for older mice. Mice younger than four weeks of age may be too excitable to provide reliable values. In order to limit the stress to the animals, it is preferable that the mice have been regularly manipulated by the investigator who will perform the test. However, no acclimatization to this test is needed. Since the test implies behavioral response, different strains may not respond equally (e.g. sv129 differ from C57BL/10ScSnJ), making the use of wild type mice of corresponding backgrounds important.

5 Methods

5.1 the “Falls and Reaches”-Method

In this method mice are subjected to a 180 sec lasting hanging test, during which a “falling” and “reaching” score is recorded. When a mouse falls or reaches one of the sides of the wire, the “falling” score or “reaching” score are diminished or increased by 1, respectively. A Kaplan-Meier-like curve can be created afterwards. For this test only a 55 cm long wire can be used described in the material section. It is of great importance for the outcome of this method that the length of the wire is remained constant between different labs, since this influences the reaches outcome.

1. Timer is set to 180 sec. The “falling” score is set to 10, and the “reaching” score is set to 0.

2. A mouse is handled by the tail and brought near the wire. The operator lets it suspend by the fore limbs only. As soon as the animal is properly suspended, the timer is started. After being released, most animals catch the wire with the four limbs. This is allowed.

3. If the timer reaches 0 sec, go to step 7.

4. If the animal reaches one end of the wire, timer is stopped and “reaching” score is increased by 1. Then, go to step 6.

5. If the animal falls, the timer is stopped, the falling score is diminished by 1 and the elapsed time is noted.

6. Provided “falling” score is >0, the procedure is restarted at step 2. If the time is over, go to step 7.

7. Test is finished. Reaching and falling scores are recorded as well as the elapsed times between falls.

The protocols allows to draw a “Kaplan-Meier-like” curve of the falls

5.2 the Longest Suspension Time-Method

A simpler protocol consists of measuring the longest suspension time in three trials. With this method an unlimited hanging time can be used or one with a fixed limit. When a fixed limit is used a hanging time of i.e. 180 or 600 seconds is recommended for old and young mdx mice respectively. Start position of the mice can also be varied; with either two or four limbs.

1. The mouse, handled by the tail, is allowed to grasp the middle of the wire with its fore limbs and is gently lowered so that its hind paws will grasp the wire a few cm apart from the fore paws.

2. The mouse is then gently accompanied while it turns upside-down along the axis of the wire.

3. The tail is released while the mouse is still grasping the wire with its four paws. Upon release, a timer is started.

As an alternative to the first three steps in this protocol, where the mouse starts with all the limbs, one could also use a start position with the two fore limbs, as is used in the “falls and reaches” method. Using the two fore limbs start position, a clear distinction can be made between mice who manage to grasp the wire with their hind limbs and those who are incapable to do so.

4. The time until the mouse completely releases its grasp and falls down is recorded.

5. The mouse is given three trials per session, with 30-sec recovery period between trials.

When a fixed time of 600 seconds is used (for mdx mice up to three months of age), mice that reach this fixed maximum, independent on the trial number, are allowed to stop with the experiment, while others are directly retested for a maximum of three times. In this case the maximum hanging time is recorded. In experiments where all mice have to hang for three trials, either the maximum hanging time or the average hanging time of the three trials can be used as an outcome measure.

For mice that are given an unlimited hanging time, the effect of body weight can be diminished by using the Holding Impulse (s*g)=Body mass (grams)×Hang Time (sec) as an outcome measure. This reflects the tension (impulse) that the animal develops for maintaining itself on the wire, against gravity for the longest period of time. When a fixed limit is used (i.e. 180 or 600 sec), the Holding impulse cannot be used, since it remains unknown what the maximum hanging time of the mice would be when no fixed time limit was used. In case none of the mice managed to hang for the fixed hanging limit, using the Holding impulse is preferred.

Body weight must be recorded either before or after the experiment. Measures may be repeated over time. However to avoid familiarization at least 1-week intervals should be used between consecutive sessions. [ . . . ]

6 Evaluation and Interpretation of Results

Both hanging wire tests described in this SOP are relative simple, reliable and are applicable to young and old mdx mice. The “falls and reaches” method is suitable to determine muscle function whereas muscle condition is measured with the longest suspension time method. [ . . . ]”

B) Clinical Trial to Assess the Effect of Nintedanib in Patients with DMD

A 52-week, double blind, randomised, placebo controlled, parallel group trial evaluating the effect of nintedanib on change in forced vital capacity (FVC) and muscular strength.

Main Inclusion Criteria: Male patients aged >12 years at visit 1 (screening); DMD diagnosis; FVC >20% predicted of normal at Visit 1 (screening)

Posology: Twice daily oral application of 25 mg, 50 mg or 150 mg nintedanib.

Primary Endpoint: Rate of change (slope) in FVC from baseline to week 52

Key Secondary Endpoint: Proportion of patients with disease progression as defined by absolute FVC (% predicted) decline ≥10% or death until week 52. Meaningful change in St. George's respiratory questionnaire (SGRQ) at week 52.

Muscular strength assessed by Quantitative Muscle Testing (QMT), North Star Ambulatory Assessment (NSAA), or the 6-min walk test (6MWT).

Safety criteria: Adverse events (especially SAE and other significant AE), physical examination, weight measurements, 12 lead electrocardiogram, vital signs and laboratory evaluations.

Statistical methods: Random coefficient regression models for continuous endpoints, Log rank tests, Kaplan-Meier plots and Cox regressions for time to event endpoints, logistic regression models or other appropriate methods for binary endpoints.

### C) Formulations for Oral Administration Containing Nintedanib

Soft gelatin capsules containing 50 mg of nintedanib

| | | Formu- | Formu- | Formu- |
|---|---|---|---|---|
| | | lationA | lation B | lation C |
| | | mg per | mg per | mg per |
| Ingredients | Function | capsule | capsule | capsule |
| Nintedanib | Active | 60.20 | 60.20 | 60.20 |
| monoethanesulphonate | Ingredient | | | |
| Triglycerides, | Carrier | 40.95 | 53.70 | 54.00 |
| medium-chain | | | | |
| Hard fat | Thickener | 38.25 | 25.50 | 25.50 |
| Lecithin | Wetting agent/ | 0.60 | 0.60 | 0.30 |
| | Glidant | | | |
| Gelatin | Film-former | 72.25 | 72.25 | 72.25 |
| Glycerol 85% | Plasticizer | 32.24 | 32.24 | 32.24 |
| Titanium dioxide | Colorant | 0.20 | 0.20 | 0.20 |
| Iron oxide red | Colorant | 0.32 | 0.32 | 0.32 |
| Iron oxide yellow | Colorant | 0.32 | 0.32 | 0.32 |
| Total Capsule Weight | | 245.33 | 245.33 | 245.33 |

Soft gelatin capsules containing 75 mg of nintedanib

| | | Formu- | Formu- | Formu- |
|---|---|---|---|---|
| | | lation A | lation B | lation C |
| | | mg per | mg per | mg per |
| Ingredients | Function | capsule | capsule | capsule |
| Nintedanib | Active | 90.3 | 90.3 | 90.3 |
| monoethanesulphonate | Ingredient | | | |
| Triglycerides, | Carrier | 61.425 | 80.55 | 80.1 |
| medium-chain | | | | |
| Hard fat | Thickener | 57.375 | 38.25 | 38.25 |
| Lecithin | Wetting agent/ | 0.9 | 0.9 | 1.35 |
| | Glidant | | | |
| Gelatin | Film-former | 107.11 | 107.11 | 107.11 |
| Glycerol 85% | Plasticizer | 46.84 | 46.84 | 46.84 |
| Titanium dioxide | Colorant | 0.35 | 0.35 | 0.35 |
| Iron oxide red | Colorant | 0.058 | 0.058 | 0.058 |
| Iron oxide yellow | Colorant | 0.16 | 0.16 | 0.16 |
| Total Capsule Weight | | 364.518 | 364.518 | 364.518 |

Soft gelatin capsules containing 150 mg of nintedanib

| | | Formu- | Formu- | Formu- |
|---|---|---|---|---|
| | | lation A | lation B | lation C |
| | | mg per | mg per | mg per |
| Ingredients | Function | capsule | capsule | capsule |
| Nintedanib | Active | 180.60 | 180.60 | 180.60 |
| monoethanesulphonate | Ingredient | | | |
| Triglycerides, | Carrier | 122.85 | 161.10 | 160.20 |
| medium-chain | | | | |
| Hard fat | Thickener | 114.75 | 76.50 | 76.50 |
| Lecithin | Wetting agent/ | 1.80 | 1.80 | 2.70 |
| | Glidant | | | |
| Gelatin | Film-former | 142.82 | 142.82 | 142.82 |
| Glycerol 85% | Plasticizer | 62.45 | 62.45 | 62.45 |
| Titanium dioxide | Colorant | 0.47 | 0.47 | 0.47 |
| Iron oxide red | Colorant | 0.08 | 0.08 | 0.08 |
| Iron oxide yellow | Colorant | 0.22 | 0.22 | 0.22 |
| Total Capsule Weight | | 626.04 | 626.04 | 626.04 |

Tablets containing 150 mg, 125 mg and 100 mg of nintedanib, respectively

| | Formulation | | Formulation |
|---|---|---|---|
| Ingredient | D | Formulation E | F |
| Nintedanib | 180.6 mg | 150.5 mg | 120.4 mg |
| monoethanesulphonate | | | |
| Lactose monohydrate | 50.0 mg | 125.0 mg | — |
| Microcrystalline cellulose | — | 20.0 mg | 150.0 mg |
| Calcium phosphate | 30.0 mg | — | 150.0 mg |
| Copovidone | 2.0 mg | 10.0 mg | — |
| Sodium starch glycolate | 5.0 mg | — | — |
| Crospovidone | — | 5.0 mg | 5.0 mg |
| Colloidal silica | 1.0 mg | 1.0 mg | 1.0 mg |
| Magnesium stearate | 4.0 mg | 4.0 mg | 4.0 mg |
| Total Tablet Weight | 272.6 mg | 315.5 mg | 430.4 mg |

Oral granules containing 125 mg of nintedanib

| Ingredient | Formulation G |
|---|---|
| Nintedanib | 150.5 mg |
| monoethanesulphonate | |
| Microcrystalline cellulose | 80.0 mg |
| Macrogol 6000 | 80.0 mg |
| Solid flavour | 5.0 mg |
| Total Weight | 315.5 mg |

The formulation may be administered in the form of granules in different amounts, e.g. containing 25 mg, 50 mg or 75 mg of nintedanib. Also, the granules can be compressed to form tablets; thus, tablets of different strengths, e.g. containing 25 mg, 50 mg or 75 mg of nintedanib, may be obtained.
Oral powder containing 50 mg of nintedanib

| Ingredient | Formulation I |
|---|---|
| Nintedanib | 60.2 mg |
| monoethanesulphonate | |
| Sorbitol | 125.0 mg |
| Microcrystalline cellulose | 20.0 mg |
| Crospovidone | 5.0 mg |
| Colloidal silica | 1.0 mg |
| Solid flavour | 4.0 mg |
| Total Weight | 215.2 mg |

The oral powder may be administered in different amounts, e.g. containing 25 mg of nintedanib.
Further pharmaceutical dosage forms for oral administration comprising nintedanib are disclosed in WO 2009/147212 and in WO 2009/147220.

1. A method for treating muscular dystrophy comprising administering a pharmaceutically effective amount of nintedanib, or a pharmaceutically acceptable salt thereof, to a patient in need thereof.

2. The method according to claim 1 wherein nintedanib is in the form of its monoethanesulfonate salt.

3. The method according to claim 1, wherein the muscular dystrophy is selected from the group consisting of Duchenne muscular dystrophy, Becker muscular dystrophy, myotonic dystrophy, facioscapulohumeral muscular dystrophy, limb-girdle muscular dystrophy, Emery-Dreifuss muscular dystrophy, congenital muscular dystrophy, oculopharyngeal muscular dystrophy, sarcoglycanopathies and dysferlinopathies.

4. The method according to claim 3, wherein the muscular dystrophy is Duchenne muscular dystrophy.

5. A method for treating muscular dystrophy comprising administering to a patient in need thereof a pharmaceutical composition comprising a pharmaceutically effective amount of nintedanib, or a pharmaceutically acceptable salt thereof, and one or more pharmaceutically acceptable excipients.

6. The method according to claim 5 wherein the pharmaceutical composition is selected from the group consisting of capsules and tablets.

7. The method according to claim 5, wherein nintedanib is in the form of its monoethanesulfonate salt.

8. The method according to claim 5, wherein the muscular dystrophy is selected from the group consisting of Duchenne muscular dystrophy, Becker muscular dystrophy, myotonic dystrophy, facioscapulohumeral muscular dystrophy, limb-girdle muscular dystrophy, Emery-Dreifuss muscular dystrophy, congenital muscular dystrophy, oculopharyngeal muscular dystrophy, sarcoglycanopathies and dysferlinopathies.

9. The method according to claim 8, wherein the muscular dystrophy is Duchenne muscular dystrophy.
